(19) United States
(12) Patent Application Publication
(10) Pub. No.: US 2022/0168371 A1
(43) Pub. Date: Jun. 2, 2022

(54) A Product and Process For A Room-Temperature Stable, Food-Grade, All-Natural, Vegan, And Phyto-Cannabinoid/Terpene/Flavonoid Colloidal Dispersion

(71) Applicant: Daniel L. Young, San Francisco, CA (US)

(72) Inventor: Daniel L. Young, San Francisco, CA (US)

(21) Appl. No.: 17/434,754

(22) PCT Filed: Mar. 4, 2020
(86) PCT No.: PCT/US20/21044

§ 371 Date: Aug. 29, 2021

Related U.S. Application Data
(60) Provisional application No. 62/813,532, filed on Mar. 4, 2019.

(51) Int. Cl. A61K 36/185; A23L 27/10; A23L 33/105; A61K 31/05; A61K 31/352; A61K 9/107

(52) U.S. Cl. CPC A61K 36/185; A23L 27/10; A23L 33/105; A61K 2236/37; A61K 31/352; A61K 9/1075; A23V 2002/00; A61K 31/05

(57) ABSTRACT

A method for producing a room-temperature stable, food-grade, all-natural, vegan, water-soluble, and phyto-cannabinoid/terpene/flavonoid colloidal dispersion by heating carrier oils and one or more than one plant extract. Mixing and heating the resultant together in a first container and adding flavor components. Mixing and heating water and one or more than one polyol in a second container. Mixing one or more than one emulsifier and one or more than one antioxidant in the second container. Mixing the contents of the first container and the second container in the first container after being removed from heat sources. Processing the resultant mixture through a high energy processor. Filtering the resultant mixture producing a room-temperature stable, food-grade, all-natural, vegan, water-soluble, and phyto-cannabinoid/terpene/flavonoid colloidal dispersion. Optionally adding an aqueous-based and lipid-based mixture to the resultant mixture that is bottled and stored in a light-protected environment and is ready for consumption.

### CROSS-REFERENCE TO RELATED APPLICATIONS

This application claims the benefit under 35 U.S.C. § 119(e) of U.S. Provisional Patent Application Ser. No. 62/813,532, filed on 2019 Mar. 4, the contents of which are incorporated herein by reference in their entirety.

### FIELD OF THE INVENTION

The present invention is in the technical field of a product and process for a room-temperature stable, food-grade, all-natural, vegan, water-soluble, and phyto-cannabinoid/terpene/flavonoid colloidal dispersion and more particularly to a room-temperature stable, food-grade, all-natural, vegan, water-soluble, and phyto-cannabinoid/terpene/flavonoid colloidal dispersion.

### BACKGROUND

Currently there is a need to formulate and produce food-grade oil-in-water emulsions that are all-natural and stable. Because certain desirable ingredients are not water soluble (i.e., lipophilic), making a formulation with them difficult and their absorption by the body very poor (i.e., low bio-availability). Emulsion-based formulations have been found to be particularly suitable for improving the oral administration of oil-soluble ingredients. These formulations are sometimes referred to as colloidal dispersions or suspensions. These emulsions are an attractive means by which to solubilize lipophilic compounds.

Problematically, oil-in-water micro-emulsions can be formulated, but often require high amounts of emulsifiers, making these formulations poor choices for food/beverage and cosmetic products. However, oil-in-water nano-emulsions are currently preferred as they typically require less emulsifier making them more suitable for food/beverage products as well as cosmetics. Keeping the emulsifier levels lower can improve flavor and also help to reduce overall cost of the formulation. Further, emulsions with small particle sizes helps to maintain the stability of the emulsion as well as enhance bio-availability of the lipophilic compounds. The small droplets in nano-emulsions provide much better stability reducing gravitational separation, flocculation, and coalescence compared to emulsions of larger particle sizes.

It has been shown that nano-emulsions can be created by using high-energy methods to break-down oil droplets into smaller particles which are stabilized by the emulsifiers which reduce the interfacial tension between the oil surface and the water phase. Examples of high-energy methods include high-pressure homogenization, micro-fluidization, and ultra-sonication. Several-low energy methods have also been shown to create nano-emulsions, such as spontaneous emulsification, phase inversion temperature, and phase inversion composition methods. However, these low-energy methods have not been extensively used in the food, beverage or cosmetics industries and their general applicability is not as well understood.

For consumption and topical uses, it is preferred that such emulsions are formulated with only all-natural, vegan, water-soluble, non-GMO, and food-grade ingredients. It is also preferred that the formulations are free of alcohol.

Currently, there are emulsions available, as shown in US 2006/0159633 A1: Emulsive water-soluble concentrates, and U.S. Pat. No. 9,743,680: Microemulsions for use in food and beverage products, but they have several shortcomings. First, the prior art does not prevent and/or reduce the oxidation of oils/lipids in the emulsion. Also, they do not formulate and create an all-natural, room-temperature stable emulsion with a hemp extract without the addition of synthetic preservatives or other extraneous natural preservatives. Additionally, they do not formulate and create an all-natural, room-temperature stable emulsion with phyto-cannabinoids, terpenes, and/or flavonoids without the addition of synthetic preservatives or other extraneous natural preservatives. Moreover, there isn't a formulation and method to incorporate a carrier oil/lipid for the “active” ingredients. There isn't a method for the addition of fat-soluble flavoring agents to the mixture before additional processing with high-energy methods. Finally, the prior art does not disclose a method for combining the resulting emulsion with a complementary or synergistic formulation that further improves the desired physiologic function.

The present invention has the right combination of all-natural ingredients that are processed to give rise to a stable emulsion with small particle sizes. The prior art teaches how to incorporate one or more synthetic emulsifier and other ingredients that are not considered all-natural. Additionally, the prior art also requires the addition of preservatives. Moreover, there is nothing in the prior art that discusses the ratio of ingredients that enables the successful processing into a stable emulsion at room temperature.

Also, there are no all-natural formulations that achieve all of the benefits of the emulsion described herein. Certain synthetic emulsifiers may be easier to use in such an emulsion, but do not meet the requirements of an all-natural formulation. Furthermore, others include preservatives, whether they be synthetic or natural, in their formulation. In addition, there are no all-natural formulations that have translucent properties due to the large size of the lipid particles in the prior art.

Therefore, there is a need for a room-temperature stable, food-grade, all-natural, vegan, water-soluble, and phyto-cannabinoid/terpene/flavonoid colloidal dispersion, overcoming the limitations of the prior art.

### BRIEF DESCRIPTION OF THE DRAWINGS

These and other features, aspects and advantages of the present invention will become better understood with regard to the following description, appended claims, and accompanying figures where:

FIG. 1 is a flowchart of some steps of a method for process for producing a room-temperature stable, food-grade, all-natural, vegan, water-soluble, and phyto-cannabinoid/terpene/flavonoid colloidal dispersion and more particularly to a room-temperature stable, food-grade, all-natural, vegan, water-soluble, and phyto-cannabinoid/terpene/flavonoid colloidal dispersion, according to one embodiment of the present invention.

### SUMMARY

The present invention solves the problems with the prior art by providing a method for producing a room-temperature stable, food-grade, all-natural, vegan, water-soluble, and phyto-cannabinoid/terpene/flavonoid colloidal dispersion. The method comprises, first heating carrier oils and one or more than one plant extract. Then, mixing the heated carrier oils and the one or more than one plant extract together in a first container. Next, optionally adding flavoring components to enhance the flavor profile of the emulsion during heating. The flavoring components are natural plant extracts that are fat-soluble. Then, mixing and heating water and one or more than one polyol in a second container. Next, mixing and heating one or more than one emulsifier and one or more than one antioxidant in the second container. Then, mixing the contents of the first container and the second container in the first container after being removed from heat sources to create and emulsion of a room-temperature stable, food-grade, all-natural, vegan, water-soluble, and phyto-cannabinoid/terpene/flavonoid colloidal dispersion. Next, processing the emulsion through a high energy processor to reduce any particulates into micro-scale or nano-scale particles for high bioavailability. Then, filtering and sterilizing the emulsion from the high energy processor to improve color, flavor and/or other aspects of the emulsion. The filtration methods, are selected considering the viscosity, particle sizes in the emulsion, desired throughout. Next, bottling the filtered emulsion, that is ready for direct consumption. Finally, storing the bottle in a light-protected environment.

Optionally, the method comprises adding one or more than one flavors including all-natural low-caloric, low-glycemic sweeteners to the mixture. Additionally, the room-temperature stable, food-grade, all-natural, vegan, water-soluble, and phyto-cannabinoid/terpene/flavonoid colloidal dispersion is combined with other secondary formulations for a variety of purposes.

Also optionally, mixing the bottled filtered resultant with a secondary formulation, wherein the secondary formulation comprises aqueous-based and lipid-based mixtures, and the secondary formulation is bottled and stored in a light-protected environment producing another room-temperature stable, food-grade, all-natural, vegan, water-soluble, and phyto-cannabinoid/terpene/flavonoid colloidal dispersion product. Inclusion of the secondary formulation is used in an alternative delivery method, such as a beverage, food, or cosmetic. Also, the secondary formulation is produced to provide complementary and/or synergistic effects on physiologic functions of the primary and secondary components. The primary and secondary component ingredients are selected from the group consisting of anti-inflammatory, mood enhancers, stress reduction, relief from vomiting/nausea, improved focus, energy, pain reduction, and sleep aids. The secondary formulation of the room-temperature stable, food-grade, all-natural, vegan, water-soluble, and phyto-cannabinoid/terpene/flavonoid colloidal dispersion further comprises colloidal silver. The secondary mixtures are aqueous or lipid-based to produce beverages, processed foods, cooked foods, lotions, pastes, creams, gels, masks, and soaps.

The dispersion is added to food, beverages, and cosmetics. The dispersion comprises a natural emulsifier, a natural carrier oil, phyto-cannabinoid/terpene/flavonoid derived from hemp, marijuana, other plants, a sugar-alcohol, and, optionally, a flavoring agent. The dispersion also comprises a concentration of one or more than one plant-derived phyto-cannabinoid compound, that can exceed 50 mg/ml, selected from the group consisting of THC, CBD, phyto-cannbinoids, terpenes and flavonoids.

The one or more than one plant extract is selected from the group consisting of synthetic and purified hemp extracts which do not contain THC, a combination of synthetic and purified extracts from different plants, and synthetic and purified natural isolates. The one or more than one carrier oil is selected from the group consisting of coconut oil, olive oil, sunflower oil, medium-chain triglycerides, natural plant oils, natural nut oils and natural seed oils, preferably medium-chain triglycerides derived from coconuts. The one or more than one fat-soluble antioxidant is selected from the group consisting of alpha-tocopherols, mixed tocopherols, vitamin E, and rosemary extracts, preferably tocopherol. The water is selected from the group consisting of deionized water, distilled water, and purified water. The one or more than one polyol are selected from the group consisting of erythritol, sorbitol, maltitol, and xylitol, preferably sorbitol. The one or more than one emulsifier is selected from the group consisting of lecithin, phospholipids, and saponins, preferably lecithin. The high-energy processor to reduce the particle sizes in the emulsion are selected from the group consisting of high-pressure homogenization, ultrasonication, and micro-fluidization.

The method further comprising the step of extracting the plant extract using a process selected from the group consisting of CO2 extraction, ethanol solvent extraction, hydrocarbon solvent extraction, distillation, decarboxylation, and combinations thereof, wherein, the one or more than one plant extract used:does not introduce any unwanted synthetic constituents; does not negatively modify plant compounds; and do not contain high levels of plant lipids/fats, chlorophyll or other constituents that could inhibit the emulsification process or lead to larger droplet sizes.

There is also provided a method for producing a room-temperature stable, food-grade, all-natural, vegan, water-soluble, and phyto-cannabinoid/terpene/flavonoid colloidal dispersion, where the first step is heating carrier oils and one or more than one plant extract. Then, mixing the heated carrier oils and the one or more than one plant extract together in a first container. Next, adding one or more than one flavor to the mixture during heating. Then, adding flavoring components to enhance the flavor profile of the emulsion during heating. Next, mixing and heating water and one or more than one polyol in a second container. Then, mixing and heating one or more than one emulsifier and one or more than one antioxidant in the second container. Next, mixing the contents of the first container and the second container in the first container after being removed from heat sources. Then, processing the resultant mixture from step g) through a high energy processor. Next, filtering the resultant mixture from the high energy processor to produce a room-temperature stable, food-grade, all-natural, vegan, water-soluble, and phyto-cannabinoid/terpene/flavonoid colloidal dispersion. Then, adding an aqueous-based and lipid-based mixture to the resultant mixture. Next, bottling the filtered resultant. Finally, storing the bottle in a light-protected environment and is ready for consumption.

The room-temperature stable, food-grade, all-natural, vegan, water-soluble, and phyto-cannabinoid/terpene/flavonoid colloidal dispersion comprises a natural emulsifier, a natural carrier oil, one or more than one phyto-cannabinoid/terpene/flavonoid derived from hemp, marijuana and/or other plants, and a sugar-alcohol. The dispersion also comprises a natural emulsifier, a natural carrier oil, one or more than one phyto-cannabinoid/terpene/flavonoid derived from hemp, marijuana and/or other plants, a sugar-alcohol, and a flavoring agent. The phyto-cannabinoid/terpene/flavonoid is lipophilic and comprises one or more than one plant-derived compound selected from the group consisting of THC, CBD, phyto-cannabinoids, terpenes and flavonoids, wherein a concentration of the phyto-cannabinoids can be varied and exceed 50 mg/ml. The processing to convert the initial mixture of ingredients into an emulsion with small particle sizes comprises a mechanical energy process selected from the group consisting of high-pressure homogenization, micro-fluidization and ultrasonication.

### DETAILED DESCRIPTION OF THE INVENTION

The present invention overcomes the limitations of the prior art by providing a room-temperature stable, food-grade, all-natural, vegan, water-soluble, and phyto-cannabinoid/terpene/flavonoid colloidal dispersion. This novel formulation and process has been invented to produce a room-temperature stable, food-grade, all-natural, vegan, water-soluble, and phyto-cannabinoid/terpene/flavonoid colloidal dispersion which can be used as a final product or added into other aqueous-based and lipid-based mixtures. The all-natural formulation incorporates a natural emulsifier, a natural carrier oil, phyto-cannabinoid/terpene/flavonoid derived from hemp, marijuana and/or other plants, a sugar-alcohol or suitable substitute, and with or without a flavoring agent, such as a plant essential oil. The phyto-cannabinoid/terpene/flavonoid, typically lipophilic, may contain one or more plant-derived compounds, including THC, CBD, and/or other phyto-cannabinoids, terpenes and flavonoids. The concentration of the phyto-cannabinoids can be varied and exceed 50 mg/ml. The processing of the emulsion involves using mechanical energy, such as with high-pressure homogenization, micro-fluidization or ultrasonication, to convert the initial mixture of ingredients into an emulsion with small particle sizes.

The methods described herein, overcome the prior art does by providing:
1) A method to prevent and/or reduce the oxidation of oils/lipids in the emulsion;
2) A method to formulate and create an all-natural, room-temperature stable emulsion with a hemp extract without the addition of synthetic preservatives or other extraneous natural preservatives;
3) A method to formulate and create an all-natural, room-temperature stable emulsion with phyto-cannabinoids, terpenes, and/or flavonoids without the addition of synthetic preservatives or other extraneous natural preservatives.
4) For the points above, a formulation and method to incorporate a carrier oil/lipid for the “active” ingredients;
5) The addition of fat-soluble flavoring agents to the mixture before additional processing with the described high-energy methods; and
6) A method for combining the resulting emulsion with a complementary or synergistic formulation that further improves the desired physiologic functions.

The method disclosed can be combined in products with phyto-cannabinoid/terpene/flavonoid that are not a macro-emulsions or nano-emulsions. This approach combines high-bioavailability phyto-cannabinoid/terpene/flavonoid emulsions, as described herein, with phyto-cannabinoid/terpene/flavonoid that has, by comparison, lower bioavailability and thus slower absorption and onset. By combining two more types of phyto-cannabinoid/terpene/flavonoid formulations, the result is that the net effects can have both rapid onset and more long-lasting effects. Namely, more rapid onset and absorption from the disclosed emulsion, combined with a slower onset and absorption from more traditional phyto-cannabinoid/terpene/flavonoid extracts/isolates. The ideal combinations would be selected for a given product and desired absorption and efficacy dynamics.

The emulsions described can be included in skincare products and topical products in general. Such as, for example, creams, lotions, serums, balms, sprays, waxes, perfumes, etc. The emulsions can also be included in personal care products, such a lubricants, condoms, etc.

The emulsions described herein can be included in un-regulated consumer products, as well as over the counter and prescription products.

A few examples of the method and the product are described herein below. As will be understood by those will skill in the art with reference to this disclosure, the following examples are intended to be illustrative of various embodiments of the present invention and are not intended to be limiting of the invention in any way.

All dimensions specified in this disclosure are by way of example only and are not intended to be limiting. Further, the proportions described herein are not necessarily to scale or proportion. As will be understood by those with skill in the art with reference to this disclosure, the actual dimensions and proportions in this disclosure will be determined by its intended use.

Methods and processes that implement the embodiments of the various features of the invention will now be described with reference to the drawings. The drawings and the associated descriptions are provided to illustrate embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

As used in this disclosure, except where the context requires otherwise, the term “comprise” and variations of the term, such as “comprising”, “comprises” and “comprised” are not intended to exclude other additives, components, integers or steps.

In the following description, specific details are given to provide a thorough understanding of the embodiments. However, it will be understood by one of ordinary skill in the art that the embodiments may be practiced without these specific details. Well-known methods, structures and techniques may not be shown in detail in order not to obscure the embodiments. For example, methods may be shown in block diagrams in order not to obscure the embodiments in unnecessary detail.

Also, it is noted that the embodiments may be described as a process that is depicted as a flowchart, a flow diagram, a structure diagram, or a block diagram. The flowcharts and block diagrams in the figures can illustrate the architecture, functionality, and operation of possible implementations of systems, methods and processes according to various embodiments disclosed. In this regard, each block in the flowchart or block diagrams can represent a segment, or portion of a process or method or implementing the invention. It should also be noted that, in some alternative implementations, the functions noted in the blocks may occur out of the order noted in the figures. Although a flowchart may describe the operations as a sequential process, many of the operations can be performed in parallel or concurrently. In addition, the order of the operations may be rearranged. A process is terminated when its operations are completed. A process may correspond to a method, a function, a procedure, etc.

In the following description, certain terminology is used to describe certain features of one or more embodiments of the invention.

The term “entourage effect” refers to the combined and synergistic effects of phyto-cannabinoids and other molecules from hemp oil all working together. Hemp extracts contain dozens of naturally occurring phytonutrients, including cannabinoids, terpenes, and flavonoids. It is desirable to formulate and produce stable all-natural, food-grade mixtures that contain one or more of compounds: phyto-cannabinoids, terpenes, and/or flavonoids. It is further desirable that such formulations enhance the bioavailability of the compounds by making them water-soluble in an oil-in-water emulsion where these compounds are contained in the tiny oil droplets. For some applications, it is further desired that the particle sizes in the emulsion are small enough to make the resulting mixture translucent. For some applications, “micro-dosing” and/a faster-acting formulation with high bioavailability is preferred to allow a more controlled delivery method. As such, an emulsion as described here may be delivered via an oral spray or oral drops in a well control fashion with micro-doses of the active constituents per spray/drop. It is preferred that the emulsion has long-term stability at room temperature. It is preferred that the emulsion can be subjected to high temperatures associated with secondary product processing, such as, for example baking or cooking. It is preferred that the emulsion can be incorporated into cosmetic products. To this end, it is preferred that the emulsion is all-natural, water-soluble and promotes absorption of beneficial compounds through the skin.

The term “flavonoids” refers to a type of polyphenol. Polyphenols are very abundant in nature and extremely diverse. There are more than 8,000 different polyphenolic compounds identified to date. Based on the differences among polyphenols, they have been subdivided into several major subclasses: phenolic acids, stilbenes, tannins, diferuloylmethanes and flavonoids. The richest sources of polyphenols in the diet include fruits, vegetables and beverages such as juices, teas, wine and coffee. The major sources of polyphenols in the average diet are flavonoids. Plants produce flavonoids as a protection against parasites, oxidative injury and harsh climatic conditions. Flavonoids are classified into subgroups based on their chemical structure: flavanones, flavones, flavonols, flavan-3-ols, anthocyanins and isoflavones. The regular consumption of flavonoids is associated with reduced risk of a number of chronic diseases, including cancer, cardiovascular disease (CVD) and neurodegenerative disorders. Their actions at the molecular level include antioxidant effects, as well the ability to modulate several key enzymatic pathways. The low solubility of flavonoids in water often presents a problem for formulation and absorption by the body of flavonoids in foods and beverages.

The term “terpene” refers to fragrant oils that give plants their aromatic diversity. Terpenes are found in many herbs, fruits, and plants, including in cannabis. Terpene molecules are volatile hydrocarbons built from repeating units of isoprene. The difference between terpenes and terpenoids is that terpenes are hydrocarbons, whereas terpenoids contain additional functional groups. For example, Vitamin A is a terpenoid. In cannabis, these terpene oils are secreted in the flower's sticky resin glands, the same ones that produce THC, CBD, and other cannabinoids. While there are more than 100 different terpenes found in cannabis, the primary terpenes include: α-pinene, linalool, myrcene, limonene, ocimene, terpinolene, terpineol, valencene, β-caryophyllene, geraniol, and humulene. Terpenes mix well with other plant extracts, concentrates and oils. Terpenes are not water soluble so they must be incorporated with other oils unless one uses an emulsifying agent.

The term “phyto-cannabinoids” refers to cannabinoids that occur naturally in the cannabis plant also known as Cannabis sativa. Cannabis sativa has been used medicinally for thousands of years; however the ways in that the cannabinoid compounds found in the Cannabis sativa plant affect the body only began to be elucidated relatively recently. Specifically, in 1988 scientists discovered receptors found on the body's cells that bind the compounds found in the Cannabis sativa plant (Devane 1988). The two main receptors found on the body's cells are now referred to as the type-1 (CB1) and type-2 (CB2) cannabinoid receptors. In addition, it was also discovered that the human body contains natural compounds that bind to these same receptors on our cells. These innate natural compounds in our body are referred to as endogenous cannabinoids, or “endocannabinoids”. When these endocannabinoids bind to these specific cell receptors, they trigger specific physiologic responses in these cells, like a lock and key system. The endocannabinoids are lipid molecules including esters, amides, and ethers of arachidonic acid (a long-chain fatty acid) which mimic the effects of THC (tetrahydrocannabinol) and other cannabinoids primarily by binding to and activating CB1 and CB2 receptors. CB1 and CB2 are G-protein coupled receptors (GPCRs) that represent the main targets of endocannabinoids. CB1 receptors are highly expressed in brain areas that control emotionality, cognition, memory, motor, and nociception (i.e., pain perception), and that includes the cortex, limbic system, hippocampus, cerebellum, and several nuclei of the basal ganglia. In fact, CB1 is the most abundant GPCR in the brain participating in neuronal plasticity. CB1 receptors are also expressed in peripheral cells and tissues, including adipose tissue, liver, and skeletal muscles. CB2 receptors are mainly present in the immune system, but are also expressed within the central nervous system (CNS), where it may play a relevant role in coping with various types of insults (Tantimonaco 2014). These natural cellular receptors and endocannabinoids in the body, in addition to several endogenous regulatory enzymes, are now referred to as the endocannabinoid system (ECS). The ECS is recognized to regulate multiple essential physiologic functions in the body, including immunity and inflammation, endocrine function, neuronal and cognitive health, digestion and appetite, mood, and pain, to name a few (Gertsch 2008, Russo 2011, Russo 2015, Russo 2016b, Izzo 2009, Jones 2012, Reekie 2017, Acharya 2017). To distinguish from endocannabinoids, the active ingredients in Cannabis sativa plant extracts are referred to as “phytocannabinoids” (where “phyto” refers to plant derived). Over 100 phytocannabinoids have now been discovered. Phytocannabinoids can be used to supplement and/or modulate the natural endocannabinoids already inside the body and help balance and/or boost the ECS. (Russo 2016b, Reekie 2017). Many of the phyto-cannabinoids were discovered in the 1960s: cannabidiol (CBD) (Mechoulam and Shvo, 1963), cannabigerol (CBG) (Gaoni and Mechoulam, 1964b), cannabichromene (CBC) (Gaoni and Mechoulam, 1966), cannabidivarin (CBDV) (Vollner et al., 1969) and tetrahydrocannabivarin (THCV) (Gill et al., 1970).

The term “all natural” refers to “nothing artificial or synthetic, including colors, regardless of source, is included in, or has been added to, the product that would not normally be expected to be there.

The term “colloidal silver” refers to a solution that can contain various concentrations of ionic silver compounds, silver colloids, or silver compounds bound to proteins in water.

Various embodiments provide a product and process for a room-temperature stable, food-grade, all-natural, vegan, water-soluble, and phyto-cannabinoid/terpene/flavonoid colloidal dispersion. One embodiment of the present invention provides a product and process for a room-temperature stable, food-grade, all-natural, vegan, water-soluble, and phyto-cannabinoid/terpene/flavonoid colloidal dispersion. The process will now be discussed in detail.

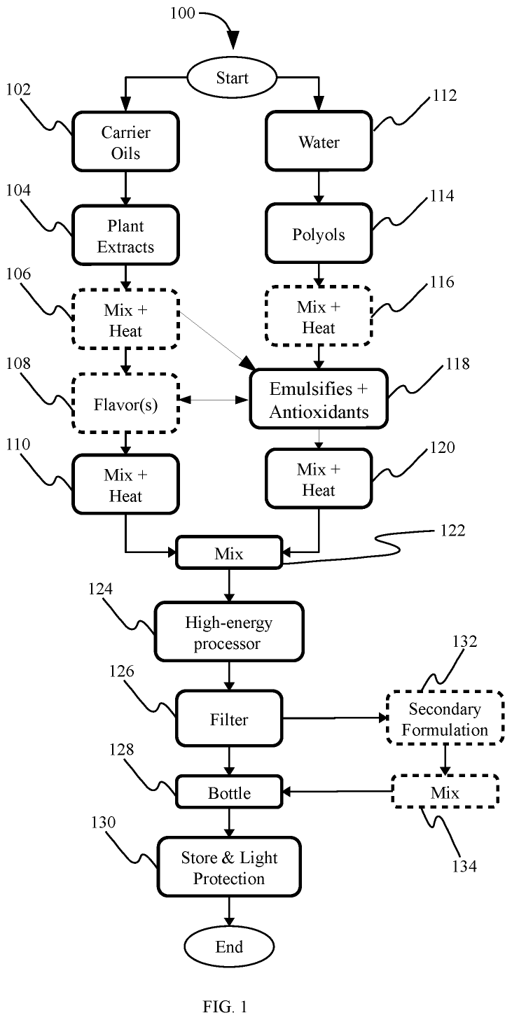

Referring to FIG. 1, there is shown a flowchart 100 of some steps of a method for a process for producing a room-temperature stable, food-grade, all-natural, vegan, water-soluble, and phyto-cannabinoid/terpene/flavonoid colloidal dispersion, according to one embodiment of the present invention.

First, carrier oils 102 and one or more than one plant extract 104 are mixed and heated together in a first container. Then, one or more than one flavor is added to the mixture and heated 106. Flavoring components 108 are mixed into and heated 108 further to enhance the flavor profile of the emulsion. Flavoring ingredients can be added such as natural plant extracts that are fat-soluble. Furthermore, all-natural low-caloric, low-glycemic sweeteners may also be added, such as monk fruit extract, stevia, etc. Next water 112 and one or more than one polyol 114 are mixed and heated 116 in a second container. Then, one or more than one emulsifier and one or more than one antioxidant 118 are mixed and heated 120 in the second container. Next, the contents of the first and second container are mixed 122 together in the first container and removed from the heat source. Then, the mixture is processed through a high energy processor 124. Suitable high energy processing hardware include: high-pressure homogenization, micro-fluidization or ultrasonication. Next, the resultant emulsion from the high energy processor 124 is filtered 126. Following the high-energy processing 124, filtration and/or sterilization 126 of the final emulsion can be performed, as required, to improve the emulsions color, flavor and/or other aspects of the emulsion. Suitable filtration methods, such as hydrophilic filter membranes, are selected considering the viscosity, particle sizes in the emulsion, desired throughout, etc. Aseptic conditions may also be used during processing to minimize the risk of contamination. Then, the filtered resultant is bottled 128. Finally, the bottle is stored 130 in a light-protected environment and is ready for consumption as a room-temperature stable, food-grade, all-natural, vegan, water-soluble, and phyto-cannabinoid/terpene/flavonoid colloidal dispersion.

Additionally, the resulting emulsion can be incorporated into secondary formulations 132 for a variety of purposes. The bottled product can be mixed 134 with a secondary formulation 132, that is then bottled and stored 130 in a light-protected environment producing a different, or second, room-temperature stable, food-grade, all-natural, vegan, water-soluble, and phyto-cannabinoid/terpene/flavonoid colloidal dispersion product.

The resulting combined formulations are preferred, over traditional approaches, given the properties of the primary emulsion: water-compatibility of the otherwise lipophilic constituents, the small particle sizes containing these constituents, the optical properties associated with emulsions of small particle sizes, and the enhanced bioavailability of these constituents in the primary formulation. Traditional approaches of incorporating such extracts do not have these advantages.

There are additional reasons for adding the primary emulsion to a secondary formulation. For example, inclusion in an alternative delivery method, such as a beverage, food, or cosmetic.

In another example, the secondary formulation has complementary and/or synergistic effects on physiologic functions of the primary and secondary components, such as ingredients that are anti-inflammatory, mood enhancers, stress reduction, relief from vomiting/nausea, improved focus/energy, pain reduction, sleep aids, etc.

An example of a novel complementary product combines our primary formulation with a secondary formulation that includes colloidal silver. See for example: https://www.ablsilver.com/Next-Generation-Colloidal-Silver. Colloidal silver has been shown to be a potent anti-inflammatory agent and natural preservative. The combination of the unique primary formulation described herein, with a colloidal silver formulation, in food, beverages, and cosmetics, presents a novel application of this technology.

The present invention produces a room-temperature stable, food-grade, all-natural, vegan, water-soluble, and phyto-cannabinoid/terpene/flavonoid colloidal dispersion which can be used as a final product or added into other aqueous-based and lipid-based mixtures, a secondary formulation. The all-natural formulation incorporates a natural emulsifier, a natural carrier oil, phyto-cannabinoid/terpene/flavonoid derived from hemp, marijuana and/or other plants, a sugar-alcohol or suitable substitute, and with or without a flavoring agent, such as a plant essential oil. The phyto-cannabinoid/terpene/flavonoid, typically lipophilic, may contain one or more plant-derived compounds, including THC, CBD, and/or other phyto-cannbinoids, terpenes and flavonoids. The concentration of the phyto-cannabinoids can be varied and exceed 50 mg/ml. The processing of the emulsion involves using mechanical energy, such as with high-pressure homogenization, micro-fluidization or ultrasonication, to convert the initial mixture of ingredients into an emulsion with small particle sizes.

Certain embodiments produce micro-scale or nano-scale particles of phyto-cannabinoid/terpene/flavonoid, and certain embodiments produce translucent emulsions. The resulting phyto-cannabinoid/terpene/flavonoid has high bioavailability, including in oral, sublingual, and stomach routes. Additional active lipophilic substances can be added with phyto-cannabinoid/terpene/flavonoid to the emulsion.

The resulting emulsions can directly be consumed via an oral spray or with a dropper into the mouth. They may also be added to other aqueous or lipid-based secondary mixtures to produce a wide variety of products, such as beverages, raw, processed or cooked foods, lotions, pastes, creams, gels, masks, and soaps (generally, foods, beverages, and cosmetics). In other embodiments, the secondary mixtures include one or more other active ingredients to complement and/or synergize with the phyto-cannabinoid/terpene/flavonoid profile.

Although there are many different possibilities and combinations that can be made from this novel method, there are presented below some preferred examples:

1) 42.4 grams of sorbitol powder are dissolved in equal parts of distilled water. 4 grams of a fraction of phospholipids from non-GMO soybeans with 70% phosphatidylcholine and 0.15% natural mixed tocopherols are dispersed into the sorbitol/water mixture. Separately, a mixture is made of 8.9 grams of coconut oil and 2.25 grams of hemp distillate. The two mixtures are combined and stirred until well mixed. The mixed solution is processed via ultra-sonication with cooling as required until the desired turbidity is reached.

2) 84.8 grams of sorbitol powder are dissolved in equal parts of distilled water. 8 grams of a fraction of phospholipids from non-GMO soybeans with 70% phosphatidylcholine and 0.15% natural mixed tocopherols are dispersed into the sorbitol/water mixture. Separately, a mixture is made of 12 grams of MCT, 6 grams of mint extract, 4.5 grams of hemp distillate. The two mixtures are combined and stirred until well mixed. The mixed solution is processed via ultra-sonication with cooling as required until the desired turbidity is reached.

There are multiple advantages to the present invention over the prior art, these advantages include:

a) a water-soluble formulation incorporating hemp/cannabis plant extracts or their constituents;

b) an all-natural formulation providing a significant market advantage for the target consumers;

c) a stable formulation providing significant advantage for distribution, shelf life, etc; and

d) a formulation with small lipid particles that improves bioavailability as well as imparting a translucent or a semi-translucent formula.

Enhanced bioavailability means that there are less active compounds to give rise to the same physiologic effects as would be needed in a different formulation with less bioavailability. This provides a competitive edge because for the same amount of active compounds one has a larger effect, or one can formulate the emulsion with less active compounds and reach the same desired physiologic effect. This also has significant cost savings for a significant competitive advantage.

Some prior art formulations use emulsifiers at very high concentrations that add cost and impart a negative flavor to the resulting emulsion. Other prior art formulations use all-natural emulsifiers, but may still require other ingredients that are not natural for preservation.

In addition to the non-limiting embodiments listed above, there are alternative all-natural ingredients and combinations, listed below, that are possible, as will be understood by those with skill in the art with reference to the present invention.

Plant extract(s) containing phyto-cannabinoids, terpenes, and/or flavonoids. Examples include: hemp extracts which do not contain THC, a combination of extracts from different plants, natural isolates. Synthetic and purified versions of the above compounds may also be used.

Carrier oil(s) including, but not limited to: coconut oil, olive oil, sunflower oil, MCT, other natural oils such as those derived from plants, nuts, and seeds.

Antioxidant (fat-soluble) including, but not limited to: alpha-tocopherols (aka, mixed tocopherols, Vitamin E, etc), and rosemary extracts.

Water, such as, for example, deionized, distilled, or purified water.

Polyol(s) or Sugar alcohols, including, but not limited to: erythritol, sorbitol, maltitol, and xylitol.

Emulsifier(s), including, but not limited to natural emulsifiers including: lecithin, phospholipids, saponins (such as from Quillaja extract).

High-energy processor to reduce the particle sizes in the emulsion including: high-pressure homogenization, ultrasonication, micro-fluidization

Preferably, the plant extracts used should not introduce any unwanted synthetic constituents. Plant extracts should not negatively modify plant compounds. Plant extracts should ideally not contain high levels of plant lipids/fats, chlorophyll or other constituents that could inhibit the emulsification process or lead to larger droplet sizes. Examples of extraction processes include: CO2 extraction, ethanol solvent extraction, hydrocarbon (Butane, Propane and Pentane) solvent extraction, distillation, etc., and combinations thereof. The extract may be further processed including decarboxylation.

Also preferably, the emulsion comprises Medium-Chain Triglycerides (MCT) that are derived from coconuts. MCT's have wide-ranging benefits, such a providing fuel for the brain and body and is easily digested and absorbed by the body.

Also preferably, Sorbitol is used as a naturally occurring sweetener for taste that is found in many fruits, berries, and vegetables, including apples and pears. Moreover, the human body produces it as part of normal metabolism. Sorbitol was first discovered in the fresh juice of mountain ash berries in 1872. In its natural state, sorbitol appears as a white crystalline powder. It is used as a common sugar substitute since it has a low glycemic index.

Also preferably, Lecithin, a natural mixture of phospholipids, is used in the emulsion as a natural substance that is essential to life. Lecithins are present in every cell in the body, and maintain cellular membrane integrity. Phospholipids also contribute to the regulation of many biological pathways, including cellular communication and synthesis of the neuro-transmitter acetylcholine, and have been shown to reduce inflammation and reduce cholesterol in certain patient populations. Phospholipids have good emulsifying properties which can stabilize emulsions. The lecithins used in the emulsion are available from several all-natural sources, including non-GMO soybeans.

Also preferably, Tocopherol is a form of Vitamin E typically derived from vegetable oils. For example, tocopherol can be derived from sunflower seed oil and non-GMO soybeans. Tocopherol is known for its antioxidant properties and helps guard against oxidation.

What has been described is a new and improved system for a room-temperature stable, food-grade, all-natural, vegan, water-soluble, and phyto-cannabinoid/terpene/flavonoid colloidal dispersion, overcoming the limitations and disadvantages inherent in the related art.

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

What is claimed is:

1. A method for producing a room-temperature stable, food-grade, all-natural, vegan, water-soluble, and phyto-cannabinoid/terpene/flavonoid colloidal dispersion, the method comprising the steps of:
a) heating carrier oils and one or more than one plant extract;
b) mixing the heated carrier oils and the one or more than one plant extract together in a first container;
c) adding flavoring components to enhance the flavor profile of the emulsion during heating,
wherein the flavoring components are natural plant extracts that are fat-soluble;
d) mixing and heating water and one or more than one polyol in a second container;
e) mixing and heating one or more than one emulsifier and one or more than one antioxidant in the second container;
f) mixing the contents of the first container and the second container in the first container after being removed from heat sources to create and emulsion of a room-temperature stable, food-grade, all-natural, vegan, water-soluble, and phyto-cannabinoid/terpene/flavonoid colloidal dispersion;
g) processing the emulsion through a high energy processor to reduce any particulates into micro-scale or nano-scale particles for high bioavailability;
h) filtering and sterilizing the emulsion from the high energy processor to improve color, flavor and/or other aspects of the emulsion;
i) bottling the filtered emulsion, that is ready for direct consumption; and
j) storing the bottle in a light-protected environment.

2. The method of claim 1, further comprising the step of adding one or more than one flavors including all-natural low-caloric, low-glycemic sweeteners to the mixture.

3. The method of claim 1, wherein the room-temperature stable, food-grade, all-natural, vegan, water-soluble, and phyto-cannabinoid/terpene/flavonoid colloidal dispersion is combined with other secondary formulations for a variety of purposes.

4. The method of claim 1, wherein the filtration methods, are selected considering the viscosity, particle sizes in the emulsion, desired throughout.

5. The method of claim 1, further comprises the step of mixing the bottled filtered resultant with a secondary formulation, wherein the secondary formulation comprises aqueous-based and lipid-based mixtures, and the secondary formulation is bottled and stored in a light-protected environment producing another room-temperature stable, food-grade, all-natural, vegan, water-soluble, and phyto-cannabinoid/terpene/flavonoid colloidal dispersion product.

6. The method of claim 5, wherein inclusion of the secondary formulation is used in an alternative delivery method, such as a beverage, food, or cosmetic.

7. The method of claim 5, wherein the secondary formulation is produced to provide complementary and/or synergistic effects on physiologic functions of the primary and secondary components.

8. The method of claim 7, where the primary and secondary component ingredients are selected from the group consisting of anti-inflammatory, mood enhancers, stress reduction, relief from vomiting/nausea, improved focus, energy, pain reduction, and sleep aids.

9. The method of claim 7, wherein the secondary formulation of the room-temperature stable, food-grade, all-natural, vegan, water-soluble, and phyto-cannabinoid/terpene/flavonoid colloidal dispersion further comprises colloidal silver.

10. The method of claim 9, wherein the room-temperature stable, food-grade, all-natural, vegan, water-soluble, and phyto-cannabinoid/terpene/flavonoid colloidal dispersion is added to food, beverages, and cosmetics.

11. The method of claim 1, wherein the room-temperature stable, food-grade, all-natural, vegan, water-soluble, and phyto-cannabinoid/terpene/flavonoid colloidal dispersion comprises a natural emulsifier, a natural carrier oil, phyto-cannabinoid/terpene/flavonoid derived from hemp, marijuana, other plants, a sugar-alcohol, and, optionally, a flavoring agent.

12. The method of claim 1, wherein the room-temperature stable, food-grade, all-natural, vegan, water-soluble, and phyto-cannabinoid/terpene/flavonoid colloidal dispersion comprises a concentration of one or more than one plant-derived phyto-cannabinoid compound, that can exceed 50 mg/ml, selected from the group consisting of THC, CBD, phyto-cannbinoids, terpenes and flavonoids.

13. The method of claim 1, wherein the secondary mixtures are aqueous or lipid-based to produce beverages, processed foods, cooked foods, lotions, pastes, creams, gels, masks, and soaps.

14. The method of claim 1, wherein:
a) the one or more than one plant extract is selected from the group consisting of synthetic and purified hemp extracts which do not contain THC, a combination of synthetic and purified extracts from different plants, and synthetic and purified natural isolates;
b) the one or more than one carrier oil is selected from the group consisting of coconut oil, olive oil, sunflower oil, medium-chain triglycerides, natural plant oils, natural nut oils and natural seed oils, preferably medium-chain triglycerides derived from coconuts;
c) the one or more than one fat-soluble antioxidant is selected from the group consisting of alpha-tocopherols, mixed tocopherols, vitamin E, and rosemary extracts, preferably tocopherol;
d) the water is selected from the group consisting of deionized water, distilled water, and purified water;
e) the one or more than one polyol are selected from the group consisting of erythritol, sorbitol, maltitol, and xylitol, preferably sorbitol;
f) the one or more than one emulsifier is selected from the group consisting of lecithin, phospholipids, and saponins, preferably lecithin; and
g) the high-energy processor to reduce the particle sizes in the emulsion are selected from the group consisting of high-pressure homogenization, ultrasonication, and micro-fluidization.

15. The method of claim 1, further comprising the step of extracting the plant extract using a process selected from the group consisting of CO2 extraction, ethanol solvent extraction, hydrocarbon solvent extraction, distillation, decarboxylation, and combinations thereof, wherein, the one or more than one plant extract used:
a) does not introduce any unwanted synthetic constituents;
b) does not negatively modify plant compounds; and
c) do not contain high levels of plant lipids/fats, chlorophyll or other constituents that could inhibit the emulsification process or lead to larger droplet sizes.

16. A method for producing a room-temperature stable, food-grade, all-natural, vegan, water-soluble, and phyto-cannabinoid/terpene/flavonoid colloidal dispersion, the method comprising the steps of:
a) heating carrier oils and one or more than one plant extract;
b) mixing the heated carrier oils and the one or more than one plant extract together in a first container;
c) adding one or more than one flavor to the mixture during heating;
d) adding flavoring components to enhance the flavor profile of the emulsion during heating;
e) mixing and heating water and one or more than one polyol in a second container;
f) mixing and heating one or more than one emulsifier and one or more than one antioxidant in the second container;
g) mixing the contents of the first container and the second container in the first container after being removed from heat sources;
h) processing the resultant mixture from step g) through a high energy processor;
i) filtering the resultant mixture from the high energy processor to produce a room-temperature stable, food-grade, all-natural, vegan, water-soluble, and phyto-cannabinoid/terpene/flavonoid colloidal dispersion;
j) adding an aqueous-based and lipid-based mixture to the resultant mixture;
j) bottling the filtered resultant; and
k) storing the bottle in a light-protected environment and is ready for consumption.

17. The method of claim 18, wherein the room-temperature stable, food-grade, all-natural, vegan, water-soluble, and phyto-cannabinoid/terpene/flavonoid colloidal dispersion comprises a natural emulsifier, a natural carrier oil, one or more than one phyto-cannabinoid/terpene/flavonoid derived from hemp, marijuana and/or other plants, and a sugar-alcohol.

18. The method of claim 18, wherein the room-temperature stable, food-grade, all-natural, vegan, water-soluble, and phyto-cannabinoid/terpene/flavonoid colloidal dispersion comprises a natural emulsifier, a natural carrier oil, one or more than one phyto-cannabinoid/terpene/flavonoid derived from hemp, marijuana and/or other plants, a sugar-alcohol, and a flavoring agent.

19. The method of claim 18, wherein the phyto-cannabinoid/terpene/flavonoid is lipophilic and comprises one or more than one plant-derived compound selected from the group consisting of THC, CBD, phyto-cannabinoids, terpenes and flavonoids, wherein a concentration of the phyto-cannabinoids can be varied and exceed 50 mg/ml.

20. The method of claim 18, wherein the step of processing to convert the initial mixture of ingredients into an emulsion with small particle sizes comprises a mechanical energy process selected from the group consisting of high-pressure homogenization, micro-fluidization and ultrasonication.
